(19) United States
(12) Patent Application Publication
(10) Pub. No.: US 2024/0269211 A1
(43) Pub. Date: Aug. 15, 2024

(54) Pharmaceutical Composition For Preventing And Treating Inflammatory Bowel Disease

(71) Applicant: Centre for Chinese Herbal Medicine Drug Development Limited, Hong Kong (CN)

(72) Inventors: Zhaoxiang BIAN, Hong Kong (CN); Chengyuan LIN, Hong Kong (CN); Haitao XIAO, Hong Kong (CN); Lixiang ZHAI, Hong Kong (CN); Hor Yue TAN, Hong Kong (CN)

(21) Appl. No.: 18/167,842

(22) Filed: Feb. 11, 2023

(51) Int. Cl. A61K 36/344; A61K 36/076; A61K 36/284; A61K 36/40; A61K 36/484; A61K 36/718; A61K 36/74; A61K 36/898; A61K 36/9066; A61P 1/12

(52) U.S. Cl. CPC A61K 36/344; A61K 36/076; A61K 36/284; A61K 36/40; A61K 36/484; A61K 36/718; A61K 36/74; A61K 36/898; A61K 36/9066; A61P 1/12

(57) ABSTRACT

The present invention belongs to the technical field of pharmaceuticals, and particularly relates to a green and healthy medicine composition for preventing and treating inflammatory bowel disease. The medicine composition comprises: 15-25 g of roots of Codonopsis pilosula (Franch.) Nannf., 5-15 g of Atractylodes macrocephala Koidz., 15-25 g of Poria cocos (Schw.) Wolf, 1-10 g of Cornus officinalis Sieb. et Zucc., 1-10 g of Coptis chinensis Franch., 1-10 g of Curcuma Longa L., 5-15 g of Paederia scandens (Lour.) Merr., 5-15 g of Bletilla striata (Thunb.) Reichb.f., and 1-10 g of Glycyrrhiza uralensis Fisch. The solution disclosed by the present application has significant beneficial effect on the prevention and treatment of inflammatory bowel disease, and the herbs used in the present application are widely used traditional Chinese medicine products with no or low toxic effect; therefore, the green and healthy medicine composition has green, healthy and safe technical advantages.

### TECHNICAL FIELD

The present invention belongs to the technical field of pharmaceuticals, and particularly relates to a green and healthy medicine composition for preventing and treating an inflammatory bowel disease.

### BACKGROUND

Inflammatory bowel disease (IBD), including ulcerative colitis (UC) and Crohn's disease (CD), is one of the most common chronic digestive diseases. According to statistics, the inflammatory bowel disease mostly occurred in western countries in the past. However, with the changes of social environment, lifestyle, dietary patterns, etc., the number of patients with the inflammatory bowel disease in China has increased significantly, and it has become one of the major digestive diseases in China. The inflammatory bowel disease mainly manifested as abdominal pain, diarrhea, bloody mucous stool and other symptoms, is mostly persistent and recurrent, can cause serious complications and induce colon cancer, and is listed as one of the modern refractory diseases by WHO. Most patients with this disease are young adults, and this disease shows a younger trend, seriously affects social productivity and personal quality of life, or even endanger life. Since the specific etiology of IBD is unclear, it is difficult to achieve radical cure by conventional drug therapy, and side effects are likely to occur. Therefore, how to find an effective and safe treatment strategy is the current hot and difficult point of IBD prevention and treatment.

At present, induction and maintenance therapies for IBD are clinically achieved mainly by aminosalicylic acids, steroids, azathioprine, biologicals, etc., but their long-term efficacy is not ideal and causes severe side effects. Therefore, safe and effective drugs with low price are urgently needed in clinical practice. Due to long research and development cycle, large investment and low success rate of new drugs; every year, there is a huge investment in the research and development of new anti-IBD drugs worldwide, but there is no breakthrough in new drugs with good clinical effects. Traditional Chinese medicines have the advantages of multiple ingredients, multiple targets, lesser side effects, better safety and low price, and have unique advantages in the prevention and treatment of complex diseases such as IBD. However, there is no clinically available, safe and reliable prescription with good efficacy.

### SUMMARY

In view of the above, the present invention provides a green and healthy medicine composition for preventing and treating an inflammatory bowel disease in order to solve the technical problems existing in the prior art.

The solution provided by the present invention for solving the technical problems thereof is that

a green and healthy medicine composition for preventing and treating an inflammatory bowel disease, characterized by comprising roots of Codonopsis pilosula (Franch.) Nannf., Atractylodes macrocephala Koidz., Poria cocos (Schw.) Wolf, Cornus officinalis Sieb. et Zucc., Coptis chinensis Franch., Curcuma Longa L., Paederia scandens (Lour.) Merr., Bletilla striata (Thunb.) Reichb.f. and Glycyrrhiza uralensis Fisch.

Preferably, the contents of the ingredients in the green and healthy medicine composition for preventing and treating the inflammatory bowel disease are: 15-25 g of roots of Codonopsis pilosula (Franch.) Nannf., 5-15 g of Atractylodes macrocephala Koidz., 15-25 g of Poria cocos (Schw.) Wolf, 1-10 g of Cornus officinalis Sieb. et Zucc., 1-10 g of Coptis chinensis Franch., 1-10 g of Curcuma Longa L., 5-15 g of Paederia scandens (Lour.) Merr., 5-15 g of Bletilla striata(Thunb.) Reichb.f. and 1-10 g of Glycyrrhiza uralensis Fisch.

Preferably, the contents of the ingredients in the green and healthy medicine composition for preventing and treating the inflammatory bowel disease are: 15 g of roots of Codonopsis pilosula (Franch.) Nannf., 5 g of Atractylodes macrocephala Koidz., 15 g of Poria cocos (Schw.) Wolf, 1 g of Cornus officinalis Sieb. et Zucc., 1 g of Coptis chinensis Franch., 1 g of Curcuma Longa L., 5 g of Paederia scandens (Lour.) Merr., 5 g of Bletilla striata (Thunb.) Reichb.f. and 1 g of Glycyrrhiza uralensis Fisch.

Preferably, the contents of the ingredients in the green and healthy medicine composition for preventing and treating the inflammatory bowel disease are: 25 g of roots of Codonopsis pilosula (Franch.) Nannf., 15 g of Atractylodes macrocephala Koidz., 25 g of Poria cocos (Schw.) Wolf, 10 g of Cornus officinalis Sieb. et Zucc., 10 g of Coptis chinensis Franch., 10 g of Curcuma Longa L., 15 g of Paederia scandens (Lour.) Merr., 15 g of Bletilla striata (Thunb.) Reichb.f. and 10 g of Glycyrrhiza uralensis Fisch.

Preferably, the contents of the ingredients in the green and healthy medicine composition for preventing and treating the inflammatory bowel disease are: 18 g of roots of Codonopsis pilosula (Franch.) Nannf., 8 g of Atractylodes macrocephala Koidz., 18 g of Poria cocos (Schw.) Wolf, 8 g of Cornus officinalis Sieb. et Zucc., 3 g of Coptis chinensis Franch., 5 g of Curcuma Longa L., 8 g of Paederia scandens (Lour.) Merr., 8 g of Bletilla striata (Thunb.) Reichb.f. and 7 g of Glycyrrhiza uralensis Fisch.

Preferably, the contents of the ingredients in the green and healthy medicine composition for preventing and treating the inflammatory bowel disease are: 20 g of roots of Codonopsis pilosula (Franch.) Nannf., 10 g of Atractylodes macrocephala Koidz., 20 g of Poria cocos (Schw.) Wolf, 5 g of Cornus officinalis Sieb. et Zucc., 5 g of Coptis chinensis Franch., 5 g of Curcuma Longa L., 10 g of Paederia scandens (Lour.) Merr., 10 g of Bletilla striata (Thunb.) Reichb.f. and 5 g of Glycyrrhiza uralensis Fisch.

### Beneficial Effect

The solution disclosed by the present application has the significant beneficial effect on the prevention and treatment of inflammatory bowel disease, and meanwhile, the herbs used in the present application are widely used traditional Chinese medicine products with no toxic effect or low toxic effect; therefore, the green and healthy medicine composition for preventing and treating the inflammatory bowel disease disclosed by the present application further has green, healthy and safe technical advantages, etc.

The technical solution and technical effect of the present application will be described in detail below with reference to the drawings and specific embodiments.

### BRIEF DESCRIPTION OF THE DRAWINGS

FIG. 1 is a comparison chart showing bloody stool score of an animal model of the first preferred embodiment;

FIG. 2 is a comparison chart showing diarrhea score of an animal model of the first preferred embodiment;

FIG. 3 is a comparison chart showing colon length of an animal model of the first preferred embodiment;

FIG. 4 is a comparison chart showing the FD4 permeability of colon tissue of the first preferred embodiment;

FIG. 5 is a comparison chart showing an inflammatory factor serum TNF-alpha level in an animal model of the first preferred embodiment;

FIG. 6 is a comparison chart showing serum IL-1beta level in an animal model of the first preferred embodiment;

LIST OF REFERENCE NUMERALS

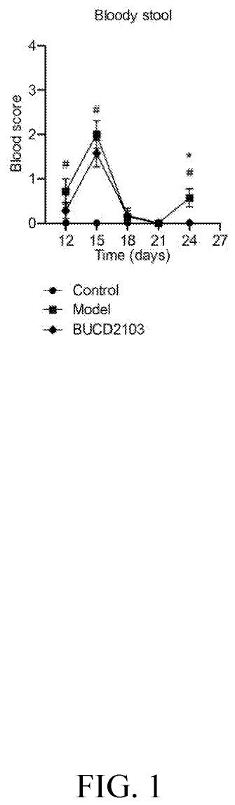

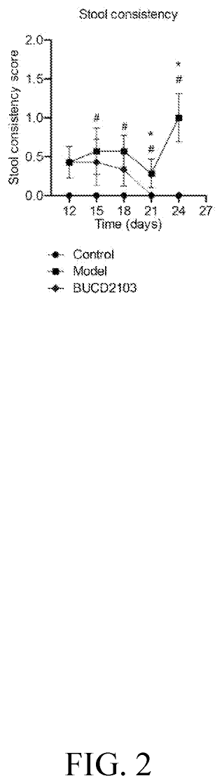

Controls in FIG. 1 and FIG. 2: blank groups;

Models in FIG. 1 and FIG. 2: model control groups;

BUCD2103 in FIG. 1 and FIG. 2: BUCD2103 prescription intervention groups; and

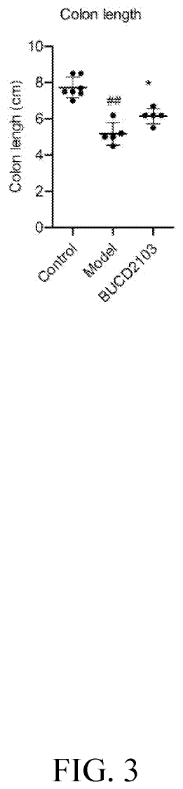

* or ** in FIG. 3-FIG. 6: BUCD2103 prescription intervention groups.

### DETAILED DESCRIPTION OF EMBODIMENTS

Pharmacodynamic experiment: A chronic inflammatory bowel disease mouse model was replicated for three cycles with 2% dextran sulfate sodium salt (DSS MW: 36,000-50,000), and meanwhile intervention was given with BUCD2103 (17.47 g dried medicinal herbs/kg. oral) at the beginning of the experiment. The classical chronic inflammatory bowel disease model was used as the experimental model of the green and healthy medicine composition for preventing and treating the inflammatory bowel disease of the present application. The modeling mice were sequentially allowed to freely drink water containing 2% DSS for 5 days, followed by distilled water for 7 days, then water containing 2% DSS for 5 days, and distilled water for 7 days, then water containing 2% DSS for 5 days, and distilled water for 7 days. By day 36, the inflammatory bowel disease mouse model was established. Later, experimental results were evaluated, compared and plotted, and comparison diagrams reflecting indexes of the inflammatory bowel disease of the model were obtained.

The prescription and experimental results of a BUCD2103 medicine composition intervention experiment are as follows:

| Number | Prescription |
|---|---|
| First preferred | 5 g of roots of Codonopsis pilosula (Franch.) Nannf., 5 g of |
| embodiment | Atractylodes macrocephala Koidz., 15 g of Poria cocos (Schw.) Wolf, |
| | 1 g of Cornus officinalis Sieb. et Zucc., 1 g of Coptis chinensis |
| | Franch., 1 g of Curcuma Longa L, 5 g of Paederia scandens (Lour.) |
| | Merr., 5 g of Bletilla striata (Thunb.) Reichb. f., and 1 g of Glycyrrhiza |
| | uralensis Fisch. |
| Second preferred | 25 g of roots of Codonopsis pilosula (Franch.) Nannf., 15 g of |
| embodiment | Atractylodes macrocephala Koidz., 25 g of Poria cocos (Schw.) Wolf, |
| | 10 g of Cornus officinalis Sieb. et Zucc., 10 g of Coptis chinensis |
| | Franch., 10 g of Curcuma Longa L, 15 g of Paederia scandens (Lour.) |
| | Merr., 15 g of Bletilla striata (Thunb.) Reichb. f. and 10 g of |
| | Glycyrrhiza uralensis Fisch. |
| Third preferred | 20 g of roots of Codonopsis pilosula (Franch.) Nannf., 10 g of |
| embodiment | Atractylodes macrocephala Koidz., 20 g of Poria cocos (Schw.) Wolf, |
| | 5 g of Cornus officinalis Sieb. et Zucc., 5 g of Coptis chinensis |
| | Franch., 5 g of Curcuma Longa L, 10 g of Paederia scandens (Lour.) |
| | Merr., 10 g of Bletilla striata (Thunb.) Reichb. f. and 5 g of |
| | Glycyrrhiza uralensis Fisch. |
| Fourth preferred | 18 g of roots of Codonopsis pilosula (Franch.) Nannf., 8 g of |
| embodiment | Atractylodes macrocephala Koidz., 22 g of Poria cocos (Schw.) Wolf, |
| | 8 g of Cornus officinalis Sieb. et Zucc., 3 g of Coptis chinensis |
| | Franch., 5 g of Curcuma Longa L, 8 g of Paederia scandens (Lour.) |
| | Merr., 8 g of Bletilla striata (Thunb.) Reichb. f. and 7 g of Glycyrrhiza |
| | uralensis Fisch. |

| | Experimental result | | | | | | | |
|---|---|---|---|---|---|---|---|---|
| | Bloody | | Diarrhea | | Colon | | | |
| Number | stool score | | score | | length | Permeability | TNF-alpha | IL-1beta |
| First preferred | 0.22 | (24 d) | 0.18 | (21 d) | 6.0 | 4450000 | 26.8 | 3.55 |
| embodiment | | | | | | | | |
| Second preferred | 0.16 | (24 d) | 0.15 | (21 d) | 6.2 | 4400000 | 26.7 | 3.72 |
| embodiment | | | | | | | | |
| Third preferred | 0 | (24 d) | 0 | (21 d) | 6.3 | 4300000 | 26.1 | 3.53 |
| embodiment | | | | | | | | |
| Fourth preferred | 0.08 | (24 d) | 0.11 | (21 d) | 6.1 | 4500000 | 27.6 | 3.64 |
| embodiment | | | | | | | | |

The principle and effect of the present application will be described below by taking the third preferred embodiment as an example. The data of the third preferred embodiment is plotted to obtain the index comparison charts of FIGS. 1-6.

Referring to FIG. 1, the experimental result of FIG. 1 indicates the effect of BUCD2103 on bloody stool score in the mouse with the chronic inflammatory bowel disease: BUCD2103 (17.47 g of dried medicinal herbs/kg, oral) can reduce the bloody stool score in the mouse with the chronic inflammatory bowel disease.

Referring to FIG. 2, the experimental result of FIG. 2 indicates the effect of BUCD2103 on diarrhea score in the mouse with the chronic inflammatory bowel disease: BUCD2103 (17.47 g of dried medicinal herbs/kg, oral) can reduce the diarrhea score in the mouse with the chronic inflammatory bowel disease.

Referring to FIG. 3, the experimental result of FIG. 3 indicates the effect of BUCD2103 on colon length in the mouse with the chronic inflammatory bowel disease: BUCD2103 (17.47 g of dried medicinal herbs/kg, oral) can inhibit colon length shortening in the mouse with the chronic inflammatory bowel disease.

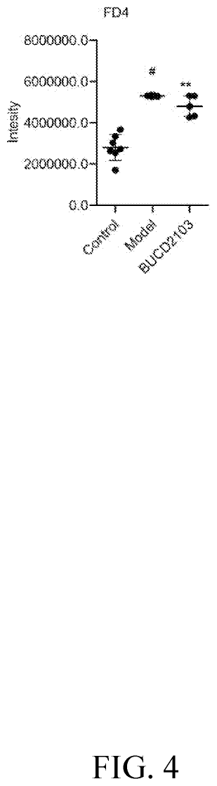

Referring to FIG. 4, the experimental result of FIG. 4 indicates the effect of BUCD2103 on the permeability of colon in the mouse with the chronic inflammatory bowel disease: BUCD2103 (17.47 g of dried medicinal herbs/kg, oral) can inhibit the FD4 permeability of the colon in the mouse with the chronic inflammatory bowel disease.

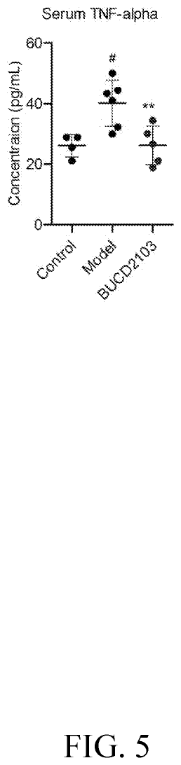

Referring to FIG. 5, the experimental result of FIG. 5 indicates that BUCD2103 (17.47 g of dried medicinal herbs/kg, oral) can inhibit the inflammatory factor TNF-alpha level of the serum in the mouse with the chronic inflammatory bowel disease.

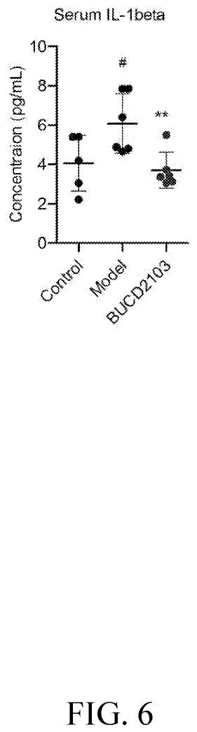

Referring to FIG. 6, the experimental result of FIG. 6 indicates that BUCD2103 (17.47 g of dried medicinal herbs/kg, oral) can inhibit the inflammatory factor IL-1beta level of the serum in the mouse with the chronic inflammatory bowel disease.

It can be seen from the above that the prescriptions disclosed in the present application all have significant beneficial effects in the prevention and treatment of inflammatory bowel disease, of which the curative effects of the third and fourth preferred embodiments are the most remarkable. Meanwhile, the herbs used in the present application are widely used traditional Chinese medicine products with no toxic effect or low toxic effect; therefore, the green and healthy medicine composition for preventing and treating the inflammatory bowel disease disclosed by the present application further has green, healthy and safe technical advantages, etc.

It should be particularly noted that the green and healthy medicine composition for preventing and treating the inflammatory bowel disease provided in the present application, the medicinal materials do not provide a simple sum of their respective efficacies, but have mutual guiding, gaining and supplementing effects according to the Monarch Minister Assistant Guide of traditional Chinese medicine. In order to prove that the medicinal materials do not provide the simple sum of their respective efficacies, the applicant used the third preferred embodiment with the most significant curative effect as a prescription, and performed a series of efficacy verification experiments according to the description in the first paragraph of Detailed Description of Embodiments. The prescription and experimental data of the efficacy verification experiments are as follows:

| Number | Prescription |
|---|---|
| Validation embodiment of roots | 90 g of roots of Codonopsis pilosula (Franch.) Nannf. |
| of Codonopsis pilosula (Franch.) | |
| Nannf. | |
| Validation embodiment of | 90 g of Atractylodes macrocephala Koidz. |
| Atractylodes macrocephala | |
| Koidz. | |
| Validation embodiment of Poria | 90 g of Poria cocos (Schw.) Wolf |
| cocos (Schw.) Wolf | |
| Validation embodiment of Cornus | 90 g of Cornus officinalis Sieb. et Zucc. |
| officinalis Sieb. et Zucc. | |
| Validation embodiment of Coptis | 90 g of Coptis chinensis Franch. |
| chinensis Franch. | |
| Validation embodiment of | 90 g of Curcuma Longa L. |
| Curcuma Longa L. | |
| Validation embodiment of | 90 g of Paederia scandens (Lour.) Merr. |
| Paederia scandens (Lour.) Merr. | |
| Validation embodiment of Bletilla | 90 g of Bletilla striata (Thunb.) Reichb. f. |
| striata (Thunb.) Reichb. f. | |
| Validation embodiment of | 90 g of Glycyrrhiza uralensis Fisch. |
| Glycyrrhiza uralensis Fisch. | |

| | Experimental result | | | | | | | |
|---|---|---|---|---|---|---|---|---|
| | Bloody | | Diarrhea | | Colon | | | |
| Number | stool score | | score | | length | Permeability | TNF-alpha | IL-1beta |
| Validation embodiment of roots | 0.84 | (24 d) | 0.30 | (21 d) | 5.1 | 5700000 | 32.2 | 5.42 |
| of Codonopsis pilosula (Franch.) | | | | | | | | |
| Nannf. | | | | | | | | |
| Validation embodiment of | 0.73 | (24 d) | 0.31 | (21 d) | 5.2 | 6400000 | 35.7 | 5.86 |
| Atractylodes macrocephala | | | | | | | | |
| Koidz. | | | | | | | | |
| Validation embodiment of Poria | 0.95 | (24 d) | 0.32 | (21 d) | 5.0 | 6300000 | 36.6 | 5.53 |
| cocos (Schw.) Wolf | | | | | | | | |
| Validation embodiment of Cornus | 0.78 | (24 d) | 0.34 | (21 d) | 4.89 | 7500000 | 37.9 | 5.66 |
| officinalis Sieb. et Zucc. | | | | | | | | |
| Validation embodiment of Coptis | 0.84 | (24 d) | 0.36 | (21 d) | 5.6 | 5900000 | 38.3 | 5.83 |
| chinensis Franch. | | | | | | | | |
| Validation embodiment of | 0.71 | (24 d) | 0.33 | (21 d) | 5.3 | 8600000 | 37.6 | 5.74 |
| Curcuma Longa L. | | | | | | | | |
| Validation embodiment of | 0.90 | (24 d) | 0.35 | (21 d) | 5.4 | 9300000 | 38.4 | 5.83 |
| Paederia scandens (Lour.) Merr. | | | | | | | | |
| Validation embodiment of Bletilla | 0.86 | (24 d) | 0.31 | (21 d) | 5.7 | 8200000 | 39.1 | 5.96 |
| striata (Thunb.) Reichb. f. | | | | | | | | |
| Validation embodiment of | 0.81 | (24 d) | 0.29 | (21 d) | 5.5 | 7900000 | 36.8 | 5.76 |
| Glycyrrhiza uralensis Fisch. | | | | | | | | |

It can be seen from the above-mentioned data that, under the same experimental conditions, the treatment of the inflammatory bowel disease using any one of the herbs in the prescription of the present application cannot produce significant efficacy, and there is a significant difference between the experimental data obtained using the prescription (the green and healthy medicine composition for preventing and treating the inflammatory bowel disease) claimed in the present application. This indicates that the medicinal materials in the present application have the mutual guiding, gaining and supplementing effects according to the Monarch Minister Assistant Guide of traditional Chinese medicine.

The technical solution and technical effect of the present invention are described above in detail with reference to the accompanying drawings and specific embodiments. It should be noted that the specific embodiments disclosed in the description are merely preferred embodiments of the present invention, and those skilled in the art can develop other embodiments on the basis thereof. Any simple modifications and equivalent substitutions made without departing from the innovation ideas of the present invention shall fall within the scope of protection of the present application.

What is claimed is:

1. A pharmaceutical composition for preventing and treating inflammatory bowel disease, comprising following components:
roots of Codonopsis pilosula (Franch.) Nannf.,
Atractylodes macrocephala Koidz.,
Poria cocos (Schw.) Wolf,
Cornus officinalis Sieb. et Zucc.,
Coptis chinensis Franch.,
Curcuma Longa L.,
Paederia scandens (Lour.) Merr.,
Bletilla striata (Thunb.) Reichb.f., and
Glycyrrhiza uralensis Fisch.

2. The green and healthy traditional Chinese medicine composition according to claim 1, comprising following components in parts by weight:
15-25 parts by weight of roots of Codonopsis pilosula (Franch.) Nannf.,
5-15 parts by weight of Atractylodes macrocephala Koidz.,
15-25 parts by weight of Poria cocos (Schw.) Wolf,
1-10 parts by weight of Cornus officinalis Sieb. et Zucc.,
1-10 parts by weight of Coptis chinensis Franch.,
1-10 parts by weight of Curcuma Longa L.,
5-15 parts by weight of Paederia scandens (Lour.) Merr.,
5-15 parts by weight of Bletilla striata (Thunb.) Reichb.f., and
1-10 parts by weight of Glycyrrhiza uralensis Fisch.

3. The green and healthy traditional Chinese medicine composition according to claim 2, comprising following components in parts by weight:
15 parts by weight of roots of Codonopsis pilosula (Franch.) Nannf.,
5 parts by weight of Atractylodes macrocephala Koidz.,
15 parts by weight of Poria cocos (Schw.) Wolf,
1 part by weight of Cornus officinalis Sieb. et Zucc.,
1 part by weight of Coptis chinensis Franch.,
1 part by weight of Curcuma Longa L.,
5 parts by weight of Paederia scandens (Lour.) Merr.,
5 parts by weight of Bletilla striata (Thunb.) Reichb.f., and
1 part by weight of Glycyrrhiza uralensis Fisch.

4. The green and healthy traditional Chinese medicine composition according to claim 2, comprising following components in parts by weight:
25 parts by weight of roots of Codonopsis pilosula (Franch.) Nannf.,
15 parts by weight of Atractylodes macrocephala Koidz.,
25 parts by weight of Poria cocos (Schw.) Wolf,
10 parts by weight of Cornus officinalis Sieb. et Zucc.,
10 parts by weight of Coptis chinensis Franch.,
10 parts by weight of Curcuma Longa L.,
15 parts by weight of Paederia scandens (Lour.) Merr.,
15 parts by weight of Bletilla striata (Thunb.) Reichb.f., and
10 parts by weight of Glycyrrhiza uralensis Fisch.

5. The green and healthy traditional Chinese medicine composition according to claim 2, comprising following components in parts by weight:
18 parts by weight of roots of Codonopsis pilosula (Franch.) Nannf.,
8 parts by weight of Atractylodes macrocephala Koidz.,
18 parts by weight of Poria cocos (Schw.) Wolf,
8 parts by weight of Cornus officinalis Sieb. et Zucc.,
3 parts by weight of Coptis chinensis Franch.,
5 parts by weight of Curcuma Longa L.,
8 parts by weight of Paederia scandens (Lour.) Merr.,
8 parts by weight of Bletilla striata (Thunb.) Reichb.f., and
7 parts by weight of Glycyrrhiza uralensis Fisch.

6. The green and healthy traditional Chinese medicine composition according to claim 2, comprising following components in parts by weight:
20 parts by weight of roots of Codonopsis pilosula (Franch.) Nannf.,
10 parts by weight of Atractylodes macrocephala Koidz.,
20 parts by weight of Poria cocos (Schw.) Wolf,
5 parts by weight of Cornus officinalis Sieb. et Zucc.,
5 parts by weight of Coptis chinensis Franch.,
5 parts by weight of Curcuma Longa L.,
10 parts by weight of Paederia scandens (Lour.) Merr.,
10 parts by weight of Bletilla striata (Thunb.) Reichb.f., and
5 parts by weight of Glycyrrhiza uralensis Fisch.
